(19) United States
(12) Patent Application Publication
(10) Pub. No.: US 2014/0288442 A1
(43) Pub. Date: Sep. 25, 2014

(54) METHOD AND APPARATUS FOR QUANTIFICATION AND MONITORING OF CARDIOVASCULAR FUNCTION DURING INDUCED STRESS OR PHYSICAL ACTIVITY AND AT REST

(71) Applicant: Tonino BOMBARDINI, Imola (Bologna) (IT)

(72) Inventor: Tonino BOMBARDINI, Imola (Bologna) (IT)

(21) Appl. No.: 14/295,117

(22) Filed: Jun. 3, 2014

Related U.S. Application Data
(63) Continuation-in-part of application No. 13/033,329, filed on Feb. 23, 2011, now abandoned.

(30) Foreign Application Priority Data
Feb. 25, 2010 (IT) BO2010A000105

(51) Int. Cl. A61B 5/0205; A61B 5/00; A61B 5/029; A61B 5/02; A61B 5/04; A61B 5/11; A61B 5/0402

(52) U.S. Cl. CPC A61B 5/0205; A61B 5/1118; A61B 5/7246; A61B 5/0402; A61B 5/02028; A61B 5/04012; A61B 5/0006; A61B 5/4884; A61B 5/029; A61B 5/02108
USPC 600/484; 600/481; 600/483; 600/508; 600/513

(57) ABSTRACT

Method and apparatus for the quantification and monitoring of cardiovascular function comprising continuous determination of significant individual cardiovascular function parameters through an operator-independent accelerometer during a sample period at rest, recording the data determined, continuously monitoring these data during pharmacological stress or exercise activity, comparing the memorized data with those determined during the same time span of the sample period and comparing the changes in cardiovascular function occurring during stress or exercise vs rest, and comparing the changes in cardiovascular function occurring during recovery vs rest and vs stress or exercise.

### CROSS REFERENCE TO RELATED APPLICATIONS

This application is a Continuation-in-Part of U.S. patent application Ser. No. 13/033,329, filed Feb. 23, 2011, the entire contents of which are incorporated by reference herein as if fully set forth.

### FIELD OF THE INVENTION

The invention relates generally to a method and the relative apparatus for monitoring the physiological conditions of an individual, with particular reference to cardiovascular function.

In particular the invention comprises a device incorporating a microprocessor, a memory and a system to control the heart and vessels, from which certain information for monitoring the cardiovascular function may derive. The cardiovascular function is monitored together with the heart rate and the activity level to establish the individual curve of the cardiovascular function variation as a function of heart rate and/or activity and to establish the individual cardiovascular function-frequency relation of the patient. In the following the terms “heart rate” and “heart frequency” have the same meaning.

### BACKGROUND

Telemonitoring of heart failure patients is a very promising way of managing several complex and costly healthcare issues. Recent European Society of Cardiology (ESC) guidelines on heart failure do recommend developing management programs for recently hospitalized patients. Screening cardiovascular function would be very important, since heart failure is associated with high morbidity, mortality, and cost. In healthy men or women, in the course of physical activity and/or pharmacological stress, left ventricular function, systemic arterial function, the right ventricular function, the function of the pulmonary circulation, and ventricular diastolic function values are generally up-sloping positive. If overt or latent cardiovascular diseases are present, this intrinsic property of the cardiovascular system is partially or totally compromised, so instead of being positive, the functional reserve is reduced, absent or becomes negative. It therefore seems crucial to develop non-invasive products to implement the ongoing monitoring of stress and/or physical activity changes in cardiovascular function

These non-invasive new sensors should be integrated with other standard known physiological sensor and biomarkers. Several strategies for monitoring cardiovascular function are used in practice.

At the present state of technology, quantification of cardiovascular function is carried out with invasive (e.g. cardiac catheterization) or non-invasive methods (e.g. myocardial scintigraphy, MRI, echocardiography) at rest or under controlled physical (exercise), pharmacological (infusion of dobutamine, dipyridamole, adenosine) or electrical (pacing) stress. These tests are frequently performed in series as using only one of them fails to provide all the necessary information for quantification of cardiovascular function.

There is considerable cost and inconvenience for the patient associated with multiple types of non-invasive measurements, and repeated cardiac catheterization. More importantly, these methods represent cardiovascular function only as one discrete point in time without the perturbance of daily activities or stress.

The proposed solutions are essentially of two types: a permanent monitoring system, reserved for more advanced disease states and which also have high costs, need for a surgical procedure for implantation; or temporary non-invasive monitoring physiological parameters, e.g. the 24-hour Holter electrocardiogram. Implantable hemodynamic monitors that are capable of measuring chronic right ventricular oxygen saturation and pulmonary artery pressure are currently being developed (Chronicle, Medtronic Inc. Minneapolis, Minn., USA).

Cardiac resynchronization therapy/defibrillators and implantable cardioverter defibrillators with continuous intrathoracic impedance monitoring capabilities (OptiVol fluid status monitoring; Medtronic Inc. Minneapolis, Minn., USA) have recently been introduced and may provide an early warning of thoracic fluid retention. However the predictive value of these implantable devices is still unknown. Furthermore, such strategies will have to be evaluated for cost effectiveness, scalability, safety, and acceptability to patients.

Wearable Sensors:

As technologies such as micro-technologies, telecommunication, low-power design, new textiles, and flexible sensors become available, new user-friendly devices can be developed to enhance the comfort and security of the patient. Since clothes and textiles are in direct contact with about 90% of the skin surface, smart sensors and smart clothes with non-invasive sensors are an attractive solution for home-based and ambulatory health monitoring. All these systems can provide a safe and comfortable environment for home healthcare, preventive medicine, and public health. A new method and a precordial non-invasive operator-independent sensor to quantify one of the parameters of the cardiovascular function has been recently patented and developed (ITA No. 01318370 dated 25 Aug. 2003) (U.S. Pat. No. 6,859,662 of Feb. 22, 2005). Expert monitoring of the heart—via this known chest wall sensor—can reliably and non-invasively sense the force-frequency relation at rest, during physical activity or during normal daily life

This known method and device (Method and device for the diagnosis and therapy of chronic heart failure, U.S. Pat. No. 6,859,662 of Feb. 22, 2005) has not the capability of monitoring the systemic blood pressure-frequency relation, the respiratory rate-frequency relation, the diastolic left ventricular active relaxation-frequency relation, the diastolic right ventricular active relaxation-frequency relation, the pulmonary artery pressure-frequency relation, the anaerobic threshold-frequency relation the recovery contractility and/or diastolic function overshoot-frequency relation.

The document U.S. Pat. No. 5,330,505 discloses implantable therapeutic devices, The U.S. Pat. No. 5,330,505 provides a method of treating a malfunctioning heart, comprising the use of many implantable sensors that are known art. To relieve the signal(s) representing the physiologic condition(s) preferably involve hemodynamic parameter(s) at the site(s) and reflect the dynamic nature of the pressure(s), O₂ saturation(s), pH(s) of blood, blood temperature(s) at the site(s) and/or interior body temperature(s), the right ventricular pulse pressure (RVPP), the right ventricular systolic pressure (RVSP) and the right atrial pressure (RAP). Arterial blood pressure also may be a particularly useful parameter; similarly, mixed venous 0₂ saturation (MVO₂) in the right atrium and/or in the right ventricle of the heart and/or elsewhere in the central venous system are useful parameters. Temperature of the interior of the patient's body also may be determined and compared against a fixed or long-term varying baseline.

The document U.S. Pat. No. 5,772,601 is focused on the pressure in which is used specifically a pressure sensor.

The invention U.S. Pat. No. 5,772,601 represents a noninvasive unique technique which is obtained by means of an external pressure transducer and separate microphones which are fixed proximally and distally of the point of the pressure transducer; this microphones are used for recording a plurality of arterial sounds while a pressure in the cuff is changed. Conclusive evidence exists based predominantly in comparison of simultaneously recorder pressocardiographic and left ventricular pressure (pressure=mmhg) curves during heart catheterization that the former mirrors in time and slope the latter. It is widely accepted that invasively obtained left ventricular pressure curves (changes of left ventricular pressure curves during cardiac cycle=changes of pressure with time=Δ pressure/Δ time) by means of micromanometer represent still today the gold standard for examining the accuracy of diastolic function assessment.

### SUMMARY

All the features of the invention so as claimed in claim 1 can be derived by one MEMS (micro electro-mechanical systems) accelerometer sensor efficiently designed and programmed to high frequency sensing and recording of multiple cardiac functions; this sensor is associated with an ECG sensor arranged to measure cardiac electrical activity and to emit electrical signals indicative of the heart rate, to build the cardiovascular function-frequency relation.

In an another embodiment of the invention, one sensor alone, without ECG reference, is utilized to monitor the heart rate and the cardiovascular function-frequency relation.

All the features claimed in claim 1 are derived by one MEMS (micro electro-mechanical systems) tri-axial accelerometer sensor efficiently designed and programmed to high frequency sensing and recording of multiple cardiac functions; this accelerometer sensor further measures the heart rate, to build the cardiovascular function-frequency relation, without ECG reference.

The tri-axial accelerometer is positioned on the chest and is efficiently arranged and programmed to record one or more cardiac functions as claimed in claim 1, the MEMS sensor continuously recording cardiac vibrations. A microprocessor automatically recognizes the pre-ejection period of each heartbeat as the peak amplitude of cardiac vibrations (first cardiac tone). The time interval (msec) occurring between two peak amplitudes of continuously recorded cardiac vibrations is calculated as the cycle duration and heart rate=60000 msec/cycle duration is monitored. The heart rate (without ECG reference) is utilized to build the cardiovascular function-frequency relation.

After that, the larger vibration signal that occurs over time between two peak amplitudes is automatically identified by the microprocessor as the second cardiac tone.

The microprocessor is able to distinguish the systolic and diastolic time length of each cardiac cycle with the assumption that at rest the first-second cardiac tone interval is shorter in comparison to the second-first cardiac tone interval.

The program of calculation applied to the processor assesses changes in values as absolute values and as percentage changes from the condition of rest to peak exercise/stress to build the cardiovascular function-frequency relationship. The calculation program can also express the cardiovascular function-frequency relation in graphic form, immediately and readily intelligible by operators of a telemetric control center, as exemplified in the following examples and figures.

The drawings show a form of the invention that is presently preferred; however, the invention is not limited to the precise arrangement shown in the drawings.

### BRIEF DESCRIPTION OF THE DRAWINGS

FIG. 1 discloses a pictorial accelerometer and related electronics;

FIG. 2 is a graph of cardiac cycle hemodynamic events with cardiac tones vibrations and temporal profiles in accordance with the present invention;

FIG. 3 is an illustration of the sensor measuring the force-frequency relation with (upper panel) the cardiac tones vibration amplitudes and (lower panel) a plot of the first cardiac tone amplitude during exercise, in accordance with embodiments of the invention;

FIG. 4 illustrates plots of instantaneous force value during exercise and recovery (left panel) and of force mobile mean related to the heart rate (right panel) in accordance with the present invention;

FIG. 5 illustrates plots of the normal up-sloping (upper left panel), flat (upper right panel), biphasic (lower left panel) force-frequency relation with a pictorial summary (lower right panel) in accordance with the present invention;

FIG. 6 illustrates plots of the sensor measured diastolic and systolic times with changing heart rates during exercise and recovery in normal (left panel) or abnormal (right panel) diastolic function;

FIG. 7 illustrates example plots of the cardiac tone built diastolic force-frequency relation in one healthy subject (upper panel) and in diastolic heart failure (lower panel) in accordance with the present invention;

FIG. 8 illustrates a plot of the instantaneous second cardiac tone force value as a function of the heart rate to derive the systemic pressure-frequency relation in accordance with the present invention;

FIG. 9 illustrates how second cardiac tone derived systemic pressure percent changes are plotted to graphically display the systemic pressure-frequency relation in accordance with the present invention;

FIG. 10 depicts graphs of hemodynamic signals versus respiration (upper panel) and graph reflecting first cardiac tone amplitude changes versus respiration (lower panel) in accordance with one embodiment as disclosed herein;

FIG. 11 shows the aortic pressure waveform (upper panels), the left ventricular first derivative of pressure changes (middle panels) and the sensor measured left ventricular active relaxation vibration amplitudes (lower panels) in a subject with normal isovolumic relaxation (left panels) or with blunted isovolumic relaxation (right panel) in accordance with embodiments of the invention;

FIG. 12 illustrates plot of instantaneous force value during exercise and recovery to measure the slope of the force-frequency relation during aerobic and anaerobic exercise and to assess the anaerobic threshold heart rate in accordance with the present invention;

FIG. 13 illustrates how the time gaps between the aortic valve and pulmonary valve opening (a,c,e) and closure (b,d,f) are liked to normal or abnormal pulmonary artery pressures to derive the pulmonary artery pressure-frequency relation in accordance with the present invention;

FIG. 14 illustrates plots (upper panel) of the normal (a) mild abnormal (b) or severe abnormal (c) pulmonary artery pressure-frequency relation and the sensor detected aortic-pulmonary valve time gaps (lower panel) in accordance with the present invention;

FIG. 15 illustrates plots of the sensor measured force-frequency relation during exercise (full symbols) and recovery (empty symbols) to establish a force recovery overshoot (left panel) in accordance with the present invention;

FIG. 16 is a schematic/block diagram illustrating an example of a cardiac tone analyzer to monitor the individual force-frequency relation and the daily activity which further communicates with a remote service center in accordance with the present invention;

FIG. 17 is a block diagram of a circuit for detecting an ECG signal to measure the heart rate, this sensor is associated with a cardiovascular function sensor to build the cardiovascular function-frequency relation in accordance with one embodiment as disclosed herein.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF THE PREFERRED EMBODIMENTS

The present invention generally concerns a method and relative apparatus for quantifying the cardiovascular function-frequency relation during stress/exercise/activity in order to implement simultaneous monitoring of multiple parameters wherein the memorized parameters comprise at least the curve of a cardiovascular function chosen between the following: the systemic blood pressure-frequency relation, the force-frequency relation, the respiratory rate-frequency relation, the diastolic left ventricular active relaxation-frequency relation, the diastolic right ventricular active relaxation-frequency relation, the pulmonary artery pressure-frequency relation, the anaerobic threshold-frequency relation, the recovery contractility and/or diastolic function overshoot-frequency relation.

Cardiovascular function values changes during stress/exercise/activity are initially identified to define the components of normality and abnormality of the individual patient. The invention provides a system comprising a microprocessor which receives information signals on the cardiovascular function.

The values are recorded at successive time units of the stress/exercise/activity, and then the system derives a plot of cardiovascular function-frequency in predetermined periods of time.

The apparatus then compares the points of the cardiovascular function-frequency relation with the memorized regions of the cardiovascular function-frequency relation in which the normal and abnormal regions are defined. The invention also comprises the apparatus for implementing the method, the essential characteristics of which are defined in the claims.

According to the invention, the cardiovascular function data are derived using sensors which emit signals indicative of the cardiovascular function.

The sensors measure the vibrations produced by the cardiovascular system and physiologically transferred from the inside of the body to the chest surface, and found here.

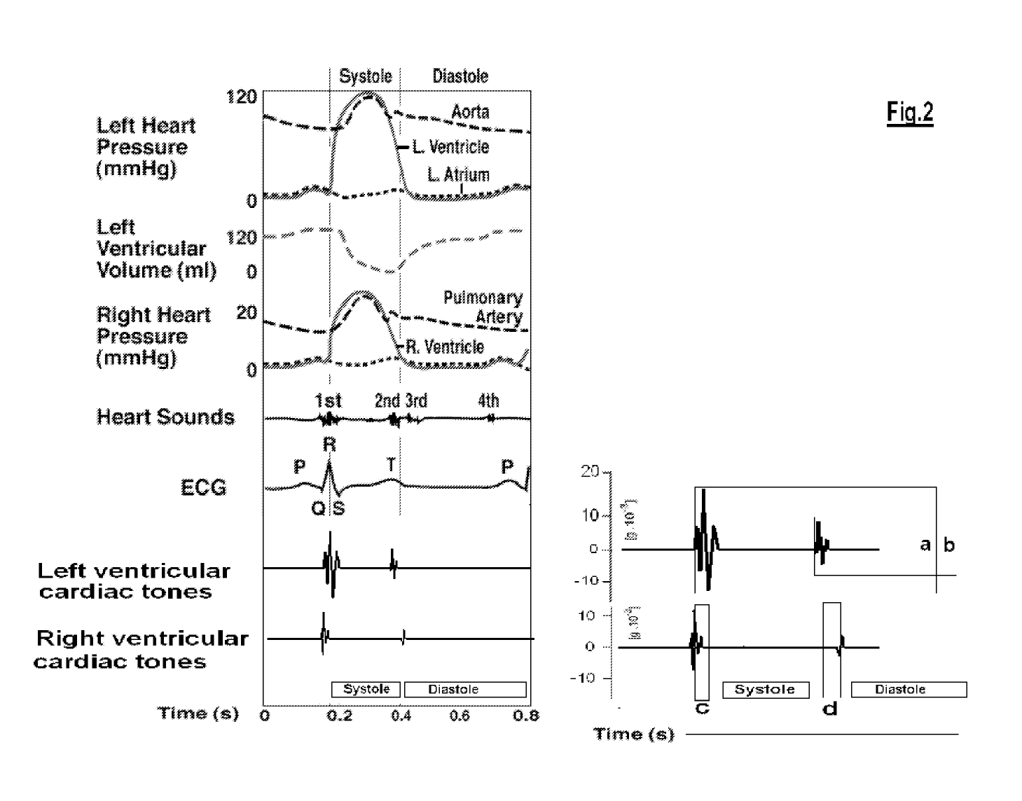

These vibrations are measured with modern accelerometer based technology (FIG. 2). The sensors quantify the amplitude, the spectral characteristics and timing of myocardial and vessels vibrations (left ventricular cardiac tones and right ventricular cardiac tones).

Vibration related signals are acquired as instantaneous values at baseline and during activity/stress: the left ventricular first cardiac tone vibration amplitude; the left ventricular second cardiac tone vibration amplitude; the right ventricular first cardiac tone vibration amplitude; the right ventricular second cardiac tone vibration amplitude; the amplitude of the vibrations generated by the aortic valve when the valve closes in the isovolumic diastole; the amplitude of the vibrations generated by the pulmonary valve when the valve closes in the isovolumic diastole; the cyclic amplitude changes of the vibrations generated by the respiratory cycle; the amplitudes of the vibrations generated by the functional aortic root-left ventricle unit during isovolumic relaxation: the amplitudes of the vibrations generated by the functional pulmonary root-right ventricle unit during isovolumic relaxation: the times between vibrations as time markers: first cardiac tone to second cardiac tone time; second cardiac tone to first cardiac tone time; and time gaps from right and left ventricular vibrations related mechanical events. The differences in amplitude and frequency clusters distinguish the different signals of the right heart from the left heart ones. For each cardiovascular function-derived parameter, the curve of the cardiovascular function variation as a function of heart rate is finally computed. The data can be also read remotely by a telemetric connection.

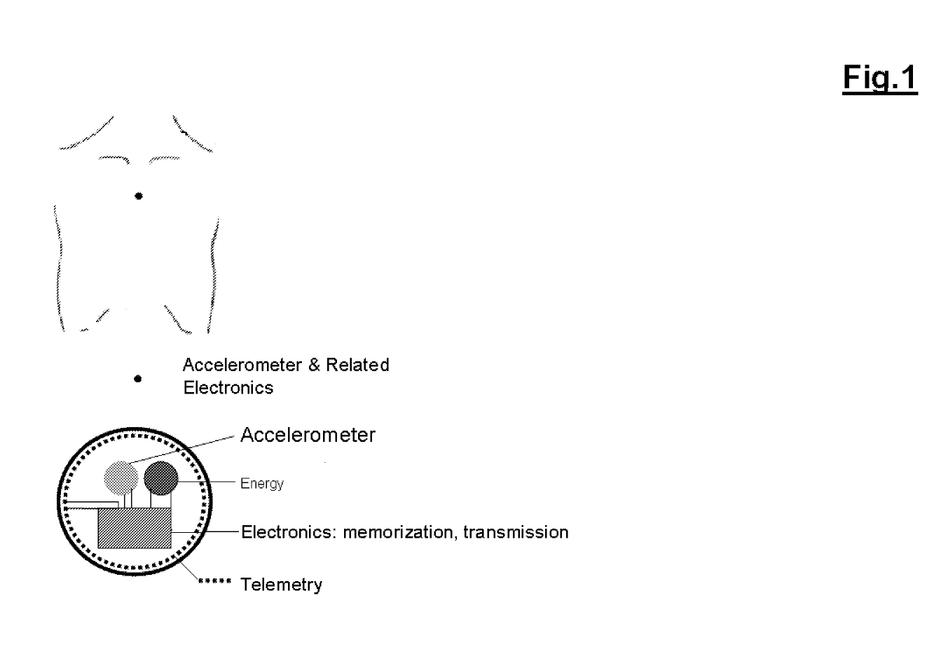

The signals emitted by said sensors are transformed from analog to digital by known means, and fed to a processor which processes them to obtain a cardiovascular function-frequency relation, and the variations thereof with time. The invention includes a data processing circuit incorporated in the apparatus that receives the electrical signals of the ECG and an accelerometer-sensor of motion. The output of the circuit can be sent to an analog to digital converter controlled by a microprocessor that converts the signals to digital data. Associated with the microprocessor there is a memory for storing digital data in an orderly manner, and these stored data can be read remotely via a telemetric connection. The microprocessor that controls the operation apparatus includes a memory containing a program of instructions executable by the microprocessor. The memory is suitable for storing digital information arriving from an analog-to-digital converter module. The invention provides an ECG sensor capable of detecting cardiac electrical activity and to emit electrical signals indicative of the heart rate, i.e. heart frequency. The ECG sensor is associated with at least one sensor indicative of cardiovascular function to build a cardiovascular function-frequency relation. The sensors can be wearable or implantable or applicable (FIG. 1).

The sensors are not electrically connected to the individual and only need to be mechanically fixed in an active or passive modality on the surface of the rib cage. Non-myocardial noise vibrations (skeletal muscles, body movements, breathing) are eliminated by frequency filtering. The sensors can also be inserted in the subcutaneous tissue, with a percutaneous procedure or in a subcutaneous pocket with a minimally surgical procedure. Said signals emitted by said sensors are transformed from analog to digital by known means, and fed to a processor that processes them to obtain a cardiovascular function-frequency relation, and the variations thereof with time.

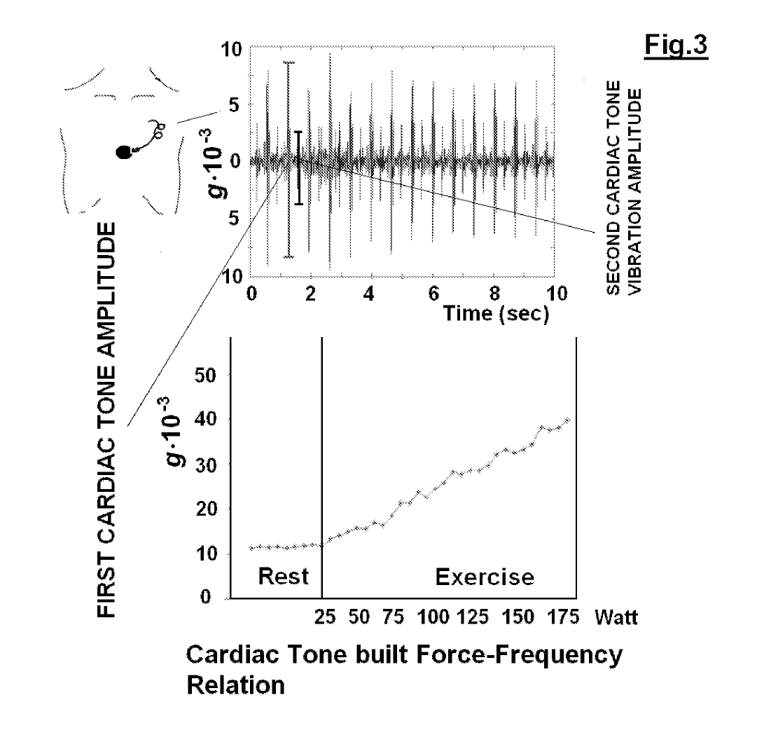

The transcutaneous force sensor is based, as said, on an accelerometer. The device includes in one single package a MEMS sensor that measures a capacitance variation in response to movement or inclination and a factory trimmed interface chip that converts the capacitance variations into analog signal proportional to the motion. The device has a full scale of ±2·g (g=9.8 m/s²) with a resolution of 0.0005·g. The transcutaneous force sensor is positioned in the mid-sternal precordial region (FIG. 3). The acceleration signal is converted to digital and recorded together with an ECG signal. The system can also be provided with a user interface that shows both the acceleration and the ECG signals while the acquisition is in progress. This sensor measures the cardiac tones generated by the myocardium during contraction (first cardiac tone) and during isovolumic relaxation (second cardiac tone) of the heart.

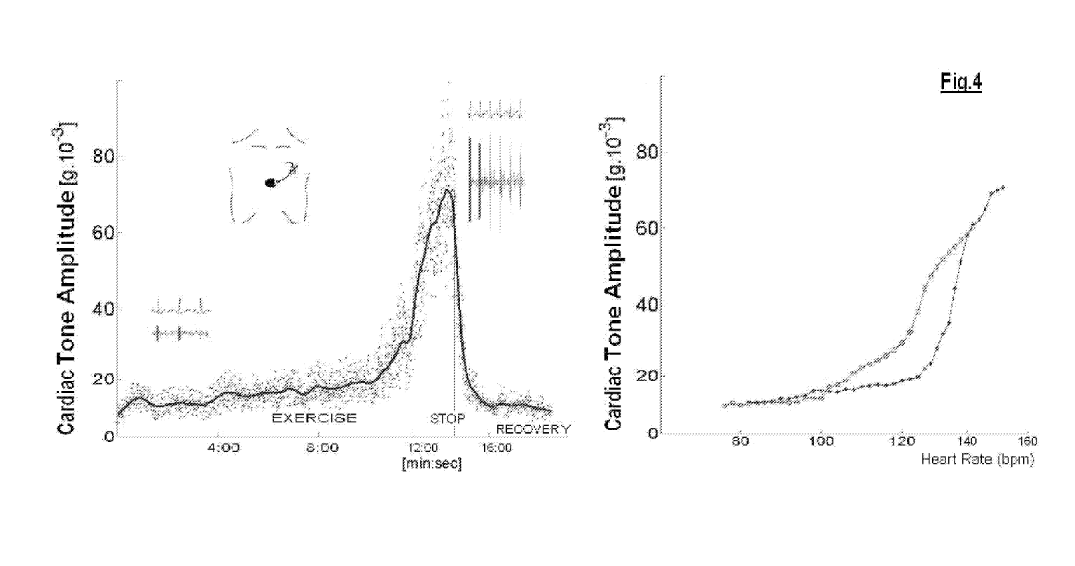

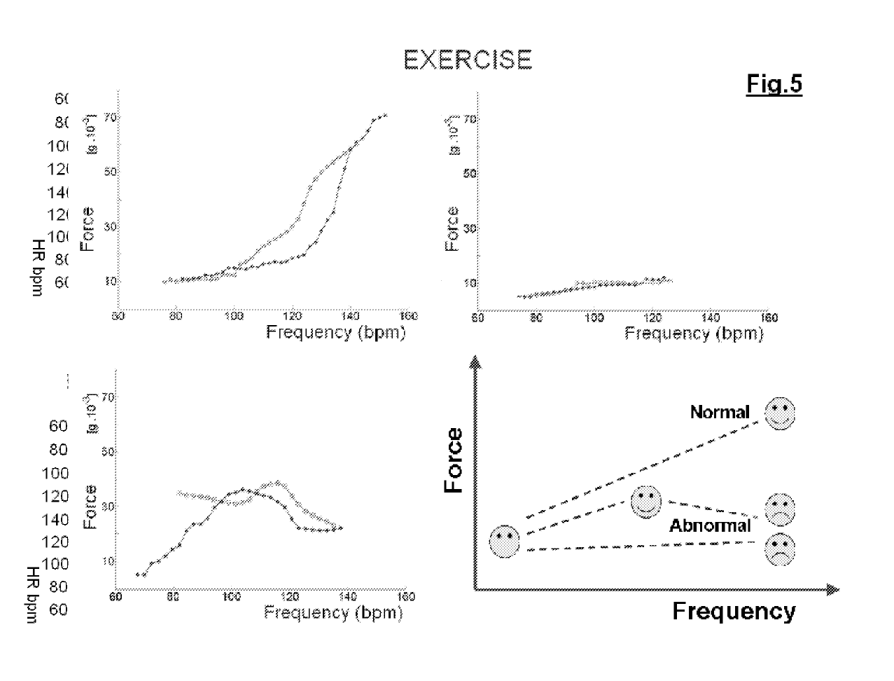

A QRS detection algorithm is used to automatically locate the beginning of the isovolumic ventricular contractions and the isovolumic relaxation of the heart. The amplitude of the vibration due to isovolumic myocardium contraction is then obtained to record the first cardiac tone amplitude as a measure of the systolic force for each cardiac beat. The curve of force variation as a function of heart rate is computed as the increment with respect to the resting force value (FIG. 4). The force-frequency relation is defined normal up-sloping when the peak stress force is higher than baseline and intermediate stress values; biphasic, with an initial up-sloping followed by a later down-sloping trend, when the peak stress force is lower than intermediate stress values; abnormal flat or negative, when the peak stress force is equal to or lower than baseline stress values (FIG. 5). The critical heart rate (or optimum stimulation frequency) is defined as the heart rate at which the force reaches the maximum value during progressive increase in heart rate; in biphasic pattern, the critical heart rate is the heart rate beyond which the force has declined by 5%; in negative pattern the critical heart rate is the starting heart rate.

The data can be also read remotely by a telemetric connection.

The sensor measures of the first cardiac tone and of the second cardiac tone are used as time markers to assess the length of expulsion time (systole) and the time of filling (diastole) of the left ventricle (FIG. 2) and the changes in systolic and diastolic times occurring with the changes in heart rate measured by the ECG.

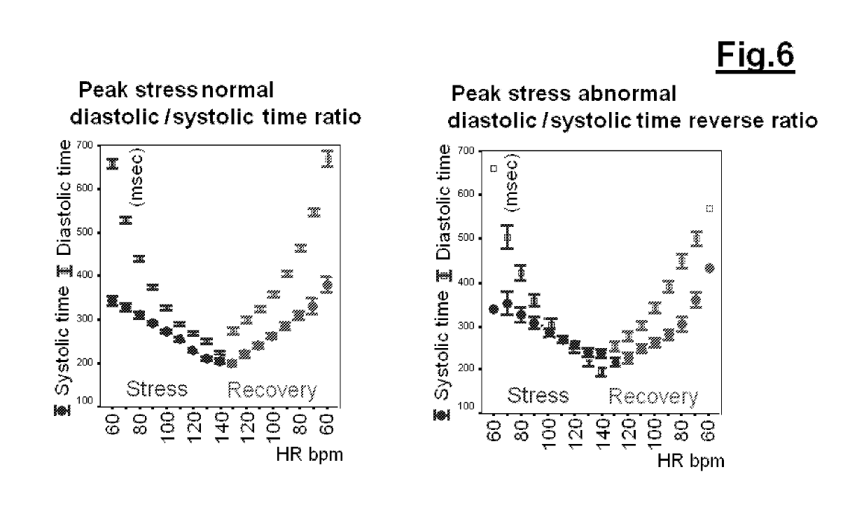

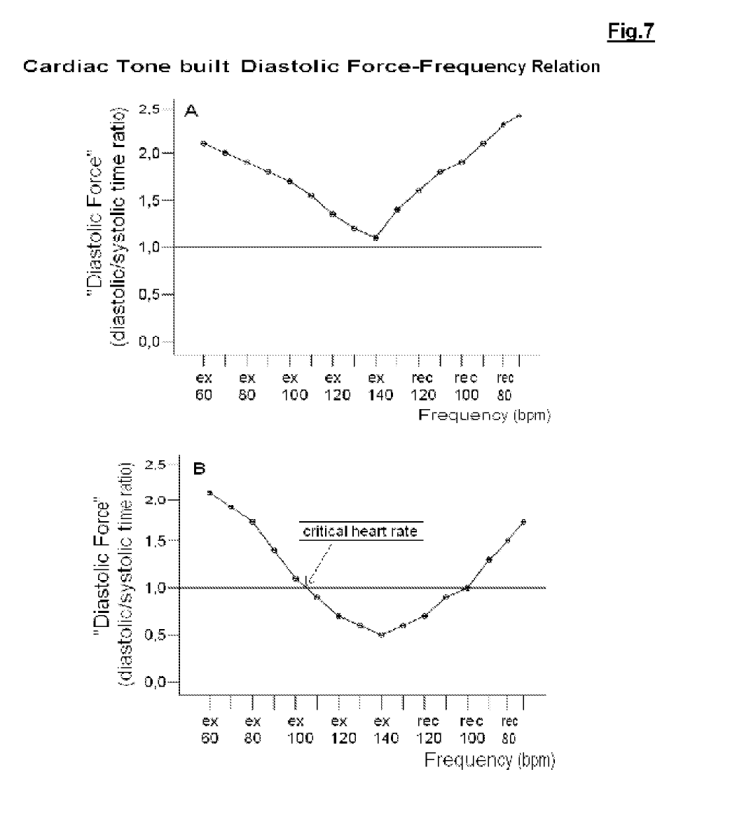

According to the physiological background, cardiac systole is demarcated by the interval between the first and the second cardiac tone, lasting from the first cardiac tone to the closure of the aortic valve. The remainder of the cardiac cycle is automatically recorded as cardiac diastole (FIG. 6). The diastolic time/systolic time ratio (the “diastolic force”) is calculated and the curve of the diastolic time/systolic time ratio variation as a function of heart rate is finally created for the quantifying of the diastolic force-frequency relation (FIG. 7).

Physiologically speaking the diastolic/systolic time ratio is a force for the heart. The heart is considered to act as a forward or force pump, serving to satisfy the augmented circulatory needs of exercise. It has long been recognized, however, that the circulation of blood during exercise must involve a two-pump system, a forward force pump and a second filling pump, which is responsible for returning blood to the heart. In considering this dual system, it is obvious that the two pumps are interdependent and must, axiomatically, generate equivalent outputs. Reversal of the normal diastolic/systolic time ratio may compromise cardiac filling and function. The systolic-diastolic force mismatch is accentuated during exercise and has the potential to impair the cardiac reserve by restricting ventricular filling and perfusion. This may have clinical implications for patients with coronary artery disease since in these cases coronary flow in mostly diastolic. At a given coronary perfusion pressure, subendocardial perfusion is dependent on the ratio between the time the heart is in diastole and the duration of systole. The diastolic/systolic time ratio indicates the duration of absence of compression of intramural vessels during a heart beat and is used as input into theoretical models on coronary perfusion. The absence of a unique relation between heart rate and diastolic/systolic time ratio on one hand and the dominant role of diastolic/systolic time ratio in subendocardial perfusion on the other hand also follow from the observation that, at the ischemic threshold, diastolic/systolic time ratio rather than heart rate correlates with the significance of coronary stenosis in patients.

The total cardiac cycle duration is algebraically dependent on the heart rate [=60,000 msec/heart rate] with fixed values totally independent from the increasing heart rate stress type.

| | HR b.p.m | | | | | | | | | | |
|---|---|---|---|---|---|---|---|---|---|---|---|
| | 50 | 60 | 70 | 80 | 90 | 100 | 110 | 120 | 130 | 140 | 150 |
| Cardiac cycle length (msec) | 1200 | 1000 | 857 | 750 | 666 | 600 | 545 | 500 | 463 | 429 | 400 |

Heart rate is the major determinant affecting diastole and systole duration. Systole is linearly related to heart rate, with the ejection time inversely related to heart rate.

However at each heart rate the fixed total cardiac cycle time can be differently divided between systole and diastole, with shorter diastolic time at higher heart rates in diseased hearts. Reversal of the normal positive (>1) diastolic/systolic ratio, as monitored by the invention, may compromise cardiac filling and function. Stress-induced “diastolic-systolic mismatch” can be easily quantified by a disproportionate decrease of diastolic time fraction, and is associated to several cardiac diseases. The diastolic force-frequency relation is defined normal when the peak stress diastolic time/systolic time ratio is positive (>1). The diastolic force-frequency relation is defined abnormal when the peak stress diastolic time/systolic time ratio is negative (<1). The diastolic force-frequency relation critical heart rate is defined as the heart rate beyond which the diastolic time/systolic time ratio from positive turns negative (FIG. 3).

The filling function of the heart is calculated by the microprocessor at rest and at peak stress as mitral filling volume divided by diastolic time (automatically sensor estimated)×1,000 and a filling function-frequency relation is monitored.

One embodiment of the invention comprises accurate measurements of the systemic and of the pulmonary mean pressure. Since the mean pressure is =systolic pressure×cardiac cycle time length/systolic time+diastolic pressure×cardiac cycle time length/diastolic time, sensor measured systolic and diastolic time length allow accurate measurement of the mean pressure at different heart rate values.

The accelerometer measures the amplitude of the vibrations generated by the aortic valve when the valve closes in the isovolumic diastole. The amplitude of these vibrations depends on the force with which the valves close, which, in turn, depends on the pressure gradient across the valve at the time of closure.

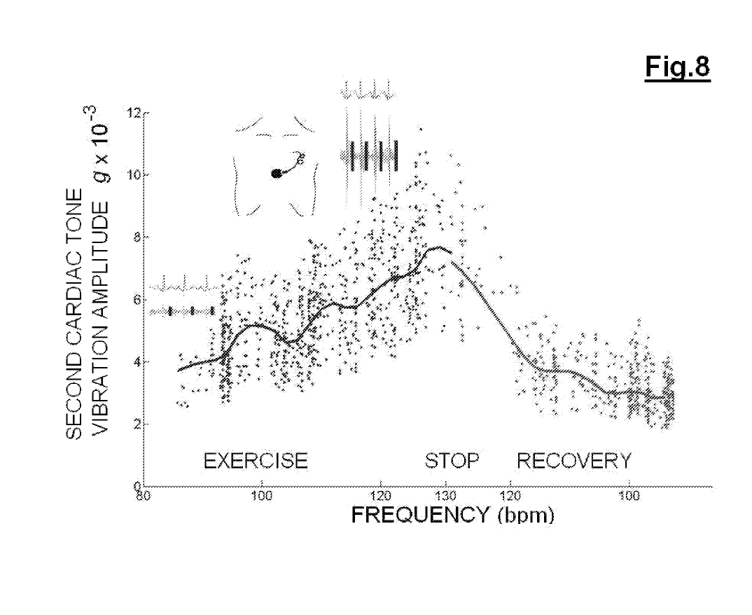

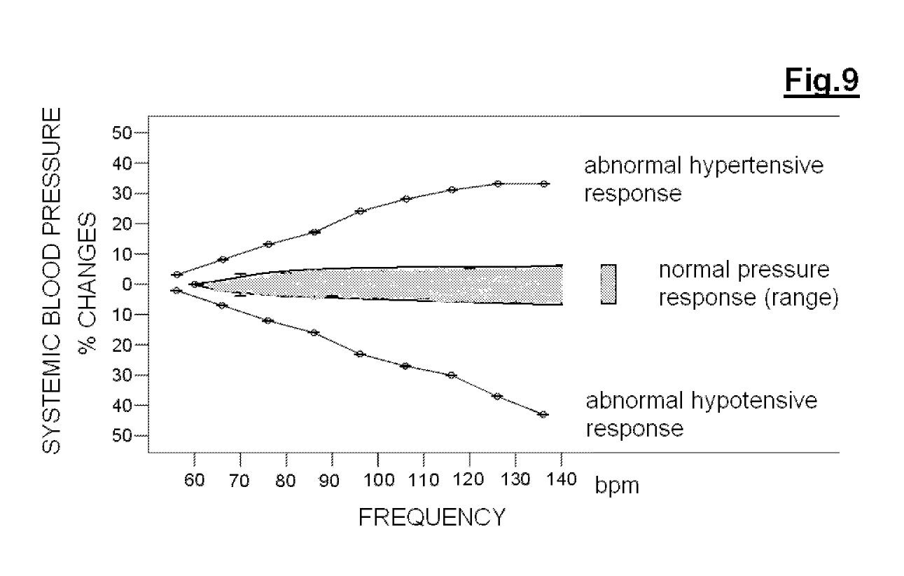

The accelerometer is positioned in the precordial region. A peak detection algorithm, synchronized with the ECG, scans the interval between the first cardiac tone and the following R wave to record the amplitude (nadir to peak) of second cardiac tone vibration for each cardiac beat (FIG. 8). This amplitude is proportional, in its variations, to changes in systemic blood pressure. An algorithm, starting from the baseline blood pressure, quantifies the changes in blood pressure over time and during changes in heart rate measured by the ECG (FIG. 9). The curve of this peak amplitude variation as a function of heart rate is finally computed as the changes with respect to the resting amplitude value. All the parameters are acquired as instantaneous values at baseline and during stress; a mobile mean is used to assess baseline, peak stress, peak-rest difference as absolute value, and delta % rest-peak stress values are computed. If the aortic pressure increases, this results in an increase both in frequency and amplitude of produced vibrations. Given a baseline systemic pressure value, an algorithm that uses the sensor output, allows the monitoring of the systemic pressure variations during stress/exercise/activity. A systemic pressure-frequency relation is constructed.

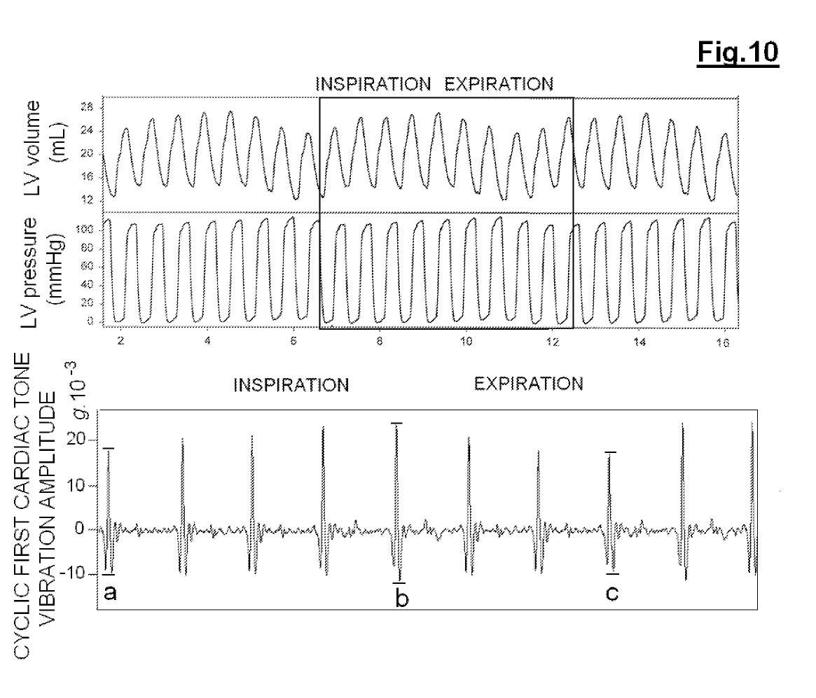

The accelerometer measures the cyclic amplitude changes of the vibrations generated by the myocardium during the pre-ejection contraction period and in the ejection period. According to the Frank-Starling law of the heart the amplitude of these vibrations shows cyclic peak amplitude variations mirroring the respiratory rate and depth of the respiratory system. These cyclic amplitude vibrations changes give the measure of the respiratory rate at rest, during activity and during recovery. The respiratory rate-frequency relation is constructed and monitored (FIG. 10). One embodiment of the invention comprises nocturnal monitoring of the respiratory rate-frequency relation to discover and quantify sleeping apnea.

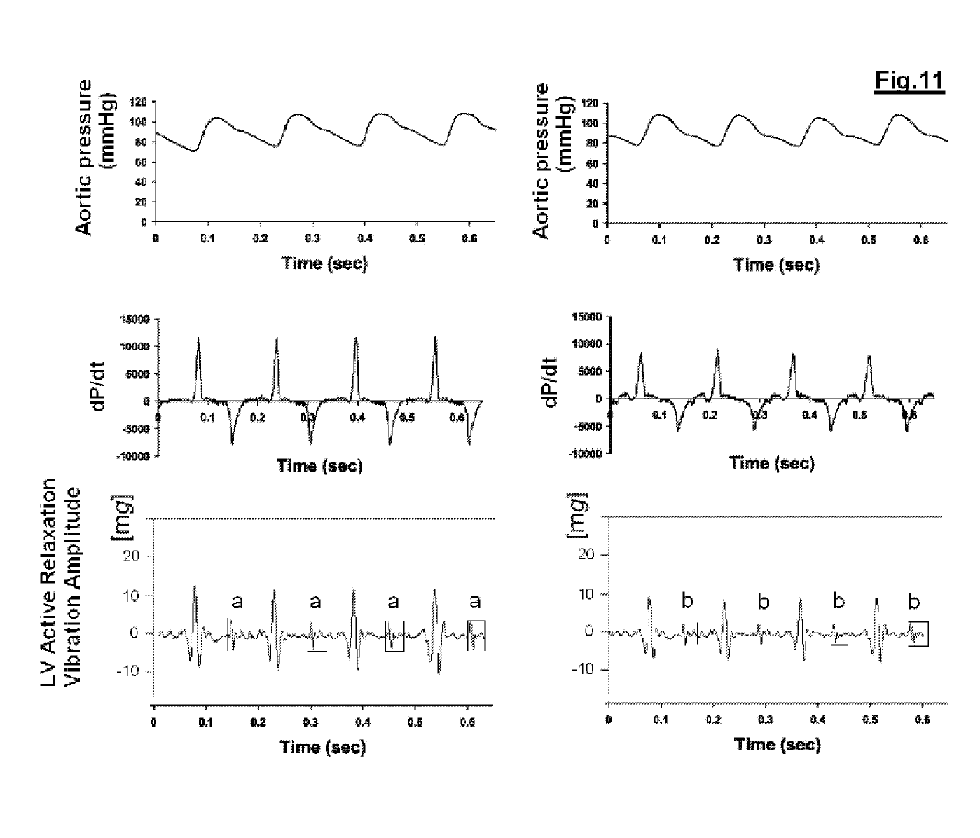

The accelerometer measures the trend of the amplitudes of the vibrations generated by the functional aortic root-left ventricle unit during isovolumic relaxation: the left ventricular isovolumic relaxation is an energy-dependent active process, and the more the process is fast and wide, more the amplitude of vibrations measured in this phase of the cardiac cycle is greater. The amplitude of these vibrations is closely linked to the driving pressure across the aortic valve (at the time of closure) and the left ventricle. Driving pressure, in the heart, refers to the instantaneous difference between arterial and ventricular pressure shortly after semilunar closure. In subjects with poor diastolic ventricular performance, the rate of isovolumic relaxation is compromised, and this causes a reduction in negative dP/dt (delta Pressure/delta time) which in turn causes a reduction of the rate of change of the pressure gradient that develops across the valve during diastole, and therefore a more slowly developing driving pressure occurs (FIG. 11). The isovolumic relaxation vibrations amplitude are acquired as instantaneous values at baseline and during stress; mobile mean is utilized to assess baseline value, at each incremental stress/exercise/activity, at peak stress/exercise/activity, and during recovery.

The curve of the diastolic isovolumic active relaxation force as a function of heart rate, i.e. the diastolic left ventricular active relaxation-frequency relation, is finally computed. The data can be also read remotely by a telemetric connection.

The accelerometer measures the trend of the amplitudes of the valvular vibration due to the driving pressure across the pulmonary valve (at the time of closure) and the right ventricle during isovolumic relaxation. Right ventricular isovolumic relaxation is an energy-dependent active process, and the more the process is fast and wide, the wider is the vibration amplitude of the right ventricular-pulmonary artery unit (at the time of closure of the pulmonary valve).

The differences in amplitude and frequency clusters distinguish the different signals of the right heart from the left heart. All the parameters are acquired as instantaneous values at baseline and during stress; mobile mean is utilized to assess baseline value, at each incremental stress/exercise/activity, at peak stress/exercise/activity, and during recovery.

The curve of the diastolic isovolumic active relaxation force, as a function of heart rate, i.e. the diastolic right ventricular active relaxation-frequency relation, is finally computed. The data can be also read remotely by a telemetric connection.

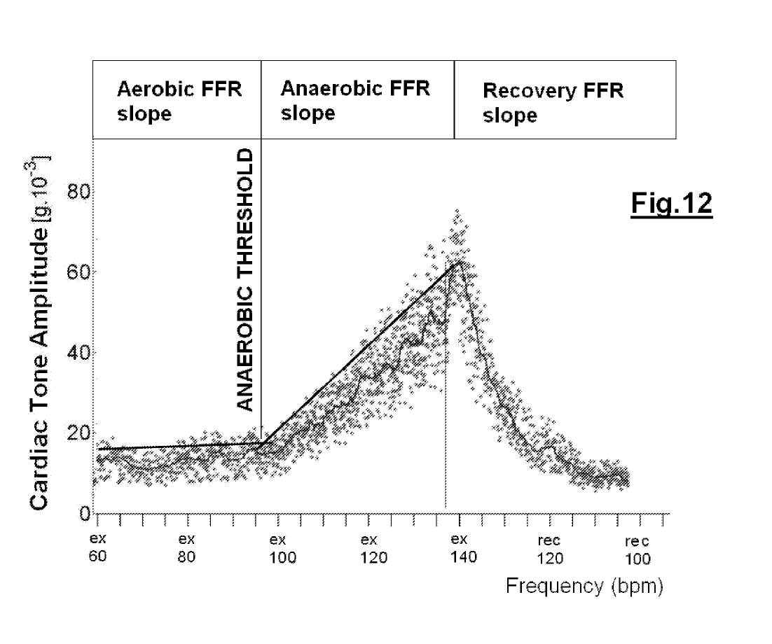

The accelerometer measures the amplitude of the vibrations generated by the myocardium during contraction. The acceleration signal is converted to digital and recorded together with an ECG signal. The sensor measures the slope of the force-frequency relation during aerobic and anaerobic exercise. In the healthy heart, during the aerobic exercise the slope of the force-frequency relation is slightly up-sloping. During anaerobic exercise, in contrast to aerobic exercise, the slope of the force-frequency relation is much steeper, and the anaerobic threshold heart rate is quantified. In the diseased heart, the anaerobic threshold occurs at lower workloads, and the anaerobic threshold heart rate is quantified (FIG. 12). A three-dimensional diagram is hence determined which not only indicates the anaerobic exercise threshold-frequency but also enables the variation of said value with time to be monitored, to assess disease progression, response to medical therapy, and improvement in cardiovascular fitness with training.

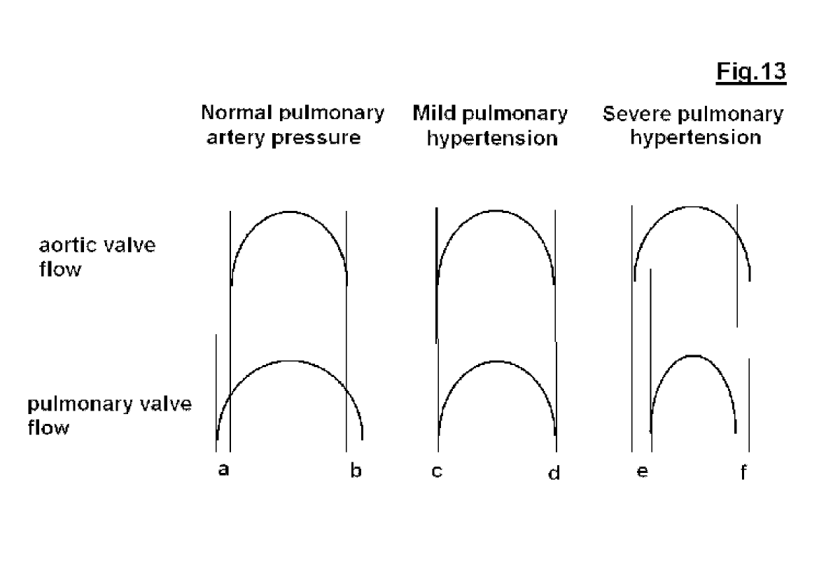

The accelerometer is used for continuous monitoring of the time gap between the aortic valve opening and pulmonary valve opening, and for continuous monitoring of the time gap between aortic valve closure and pulmonary valve closure. The vibration frequencies present in aortic and pulmonary valve closure and opening in the cardiac cycle are determined by the volume of the vibrating mass (smaller volume has a higher resonance frequency) and the tension generated in the walls of the heart and great vessels. Which easily distinguish vibrations within the aorta and pulmonary artery. The time gap between aortic valve opening and pulmonary valve opening, and the time gap between aortic valve closure and pulmonary valve closure, are strictly related to normal vs abnormal changes in pulmonary artery pressure. In the normal heart (FIG. 13) the pulmonary valve opens before and closes after the aortic valve. When mild pulmonary hypertension occurs, the pulmonary valve opens and closes simultaneously with the aortic valve. When severe pulmonary hypertension occurs, the pulmonary valve opens before and closes after the aortic valve.

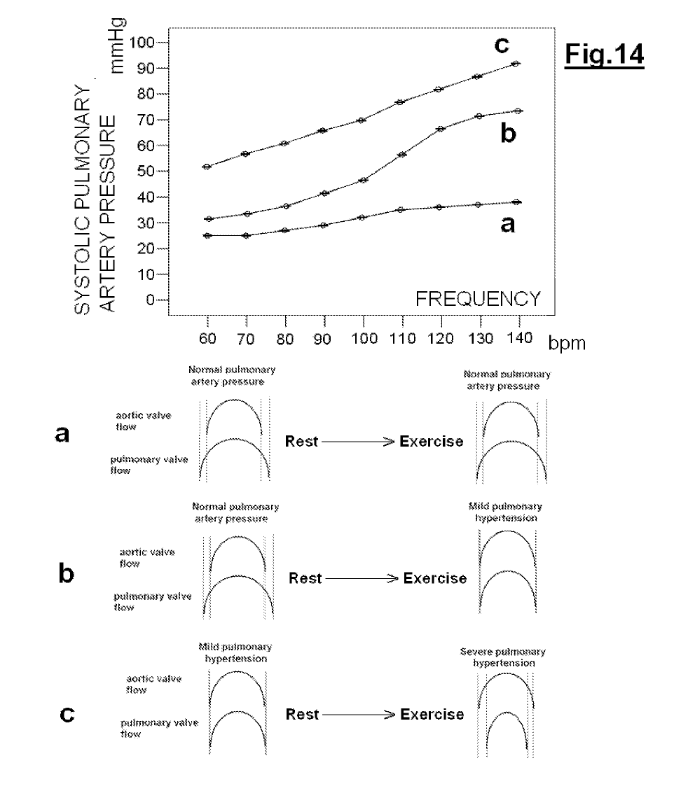

Given a basal pulmonary pressure rest value (preferably by inserting a baseline measured hemodynamic or Doppler derivative measure), an algorithm, based on the time gap between aortic valve and pulmonary valve opening and closure, easily calculates the pulmonary pressure values during activity, peak stress, and recovery. The pulmonary artery pressure-frequency relation is computed (FIG. 14).

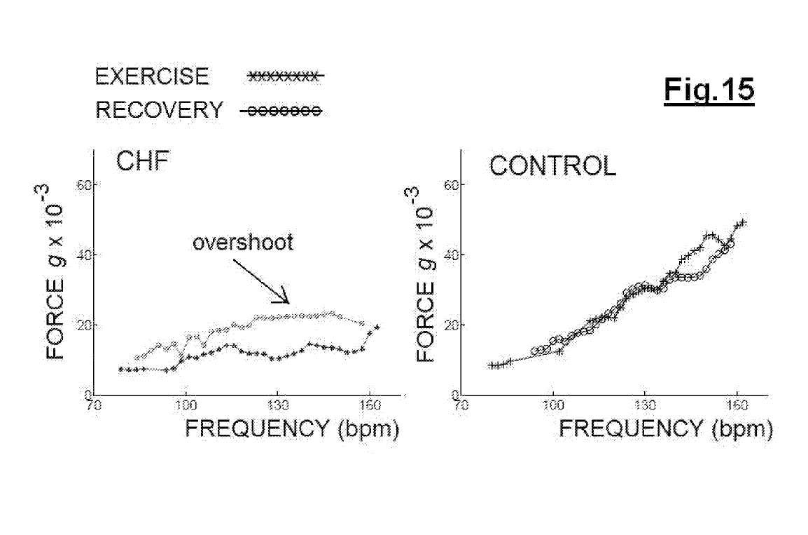

The accelerometer measures the amplitude of the vibrations generated by the myocardium during contraction (a measure of ventricular force) and during isovolumic relaxation of the heart: these vibrations are used to monitor the systolic force-frequency relation and diastolic force-frequency relation. Then a comparator compares the values of systolic force and of diastolic force at the same heart rate values during stress/exercise/activity towards recovery. The post-exercise systolic force overshoot (defined as a relative increase in recovery systolic force of more than 10% with respect to the exercise/activity value) is recorded with the cutaneous sensor and frequently associated with an abnormal blunted force-frequency relation during exercise: the contractile overshoot is a compensatory phenomenon in heart failure and post-myocardial infarction disease (FIG. 15).

The post-exercise diastolic force overshoot (defined as a relative increase in recovery diastolic/systolic time ratio of more than 10% with respect to the exercise value, is associated with better filling of the heart and with increased coronary artery perfusion time (the coronary flow is almost totally diastolic) (FIG. 6).

### Example 1

### Shown in FIG. 1

Sensorized Textiles and Portable Sensor

Wearable sensor in direct contact with the chest skin surface, Expert monitoring of the heart—via a chest wall sensor—can reliably and non-invasively sense the force-frequency relation at rest, during physical activity or during normal daily life. The sensors of the present invention may be affixed to the body of a patient by any known means. For example, the sensor that require intimate contact with the exterior of a particular area of the body may be held in place by various types of flexible belts, elastic wraps, dermal adhesives, elastic textiles, and the like.

The sensor monitors the systolic and diastolic force-frequency relation, the systemic blood pressure-frequency relation, the respiratory rate-frequency relation, the diastolic left ventricular active relaxation-frequency relation, the diastolic right ventricular active relaxation-frequency relation, the anaerobic threshold-frequency relation, the pulmonary artery pressure-frequency relation, contractility and/or diastolic function overshoot in the recovery period.

### Example 2

### Shown in FIG. 2

Cardiac Tones Accelerations Signals and Cardiac Cycle Hemodynamic Events

Left: the different phases of the cardiac cycle in which the sensor quantifies the amplitude, the spectral characteristics and timing of myocardial and vessels vibrations (left ventricular cardiac tones and right ventricular cardiac tones).

Right: the amplitude of the vibration signals (a=first cardiac tone vibration amplitude; b=second cardiac tone vibration amplitude) the times between vibrations as time markers (c and d=time gaps from right and left ventricular vibrations related mechanical events) are acquired as instantaneous values at baseline and during activity/stress. For each cardiovascular function-derived parameter, the curve of the cardiovascular function variation as a function of heart rate is finally computed. The data can be also read remotely by a telemetric connection.

### Example 3

### Shown in FIG. 3

The Accelerometer for Measuring the Force-Frequency Relation

Upper panel: X-axis: time (sec); Y-axis: cardiac tone vibration amplitude, g×10⁻³ (g=9.8 m/sec²).

The accelerometer is positioned in the mid-sternal precordial region. This sensor measures the cardiac tones generated by the myocardium during contraction (first cardiac tone) and during isovolumic relaxation (second cardiac tone) of the heart. A QRS detection algorithm is used to automatically locate the beginning of the isovolumic ventricular contractions.

Lower panel: X-axis: exercise workload (Watt); Y-axis: first cardiac tone vibration amplitude during exercise, g×10⁻³ (g=9.8 m/sec²).

The amplitude of the vibration due to isovolumic myocardium contraction is obtained to record the first cardiac tone amplitude as a measure of the systolic force; for each cardiac beat the parameters are acquired as instantaneous values at rest and during exercise/stress/activity.

### Example 4

### Shown in FIG. 4

The Curve of First Cardiac Tone Amplitude as a Function of Heart Rate

Left panel. X-axis: time, (sec); exercise=exercise in progress; recovery=recovery from exercise. Y-axis: first cardiac tone vibration amplitude, g×10⁻³ (g=9.8 m/sec²). All the parameters are acquired as instantaneous values at baseline, during exercise and recovery. Mobile mean is utilized to assess baseline value (1 minute recording), at each incremental stress test, at peak test, and during recovery. Instantaneous force values scattering (points) depend on the respiratory cycle and thorax expansion; continuous line=force mobile mean.

Right panel. X-axis: heart rate, beats per minute (bpm); Y-axis: first cardiac tone vibration amplitude, g×10⁻³ (g=9.8 m/sec²).

Full symbols=exercise in progress; empty symbols=recovery.

### Example 5

### Shown in FIG. 5

Sensor Built Force-Frequency Relation

X-axis: Frequency=heart rate, beats per minute (bpm); Y-axis: Force=first cardiac tone vibration amplitude, g×10⁻³ (g=9.8 m/sec²).

Full symbols=exercise in progress; empty symbols=recovery from exercise.

Left upper panel, normal up sloping force-frequency relation: the Δ rest-peak force is >15.5 g*10⁻³ (cut-off value for normal contractile reserve). Right upper panel, abnormal flat force-frequency relation: the Δ rest-peak force is 5 g*10⁻³, much less than 15.5 g*10⁻³ (cut-off value for normal contractile reserve).

Lower left panel, the force-frequency relation is biphasic, with an initial up-sloping trend followed by a later down-sloping trend: the critical heart rate occurs at 110 bpm. The critical heart rate (or optimum stimulation frequency) is the human counterpart of the treppe phenomenon in isolated myocardial strips; the optimal heart rate is not only the rate that would give maximal mechanical performance of an isolated muscle twitch, but also is determined by the need for diastolic filling.

Lower right panel: The force-frequency relationship is defined up-sloping when the peak exercise force is higher than baseline and intermediate stress values; biphasic, with an initial up-sloping followed by a later down-sloping trend, when the peak exercise force is lower than intermediate stress values; flat or negative, when the peak exercise force is equal to or lower than baseline values. In biphasic pattern, the critical heart rate is the heart rate beyond which the force has declined by 5%; in negative pattern the critical heart rate is the starting heart rate.

### Example 6

### Shown in FIG. 6

Sensor-based diastolic and systolic times measurement during stress and recovery

X-axis: heart rate bpm, (beats per minute); Y-axis: diastolic and systolic times (msec)

Diastolic (empty symbols) and systolic (full symbols) times at increasing heart rates during exercise (stress) and at decreasing heart rates during recovery (recovery).

Left panel: at peak stress a normal diastolic time is still longer than systole.

Right panel: at peak stress a reversal of the diastolic/systolic time ratio occurs, with the duration of systole longer than diastole. The diastolic-systolic mismatch, with relative systolic dominance, is promptly resolved during recovery. At each recovery heart rate the diastolic time increases with respect to the exercise period (both in the left and in the right panel) with a recovery diastolic time overshoot. Diastolic time overshoot is observed at each recovery heart rate, with improved ventricular filling and coronary perfusion time.

### Example 7

### Shown in FIG. 7

Sensor Built Diastolic Force-Frequency Relation

X-axis: heart rate bpm, (beats per minute); ex=exercise in progress; rec=recovery from exercise

Y-axis: diastolic force expressed as diastolic/systolic time ratio at increasing heart rates during exercise, and at decreasing heart rates during recovery.

According to the physiological background, cardiac systole is demarcated by the interval between the first and the second cardiac tone, lasting from the first cardiac tone to the closure of the aortic valve. The remainder of the cardiac cycle is automatically recorded as cardiac diastole. The diastolic time/systolic time ratio (the “diastolic force”) is calculated and the curve of the diastolic time/systolic time ratio variation as a function of heart rate is finally created for the quantifying of the diastolic force-frequency relation.

Upper panel: The curve of the diastolic force variation as a function of heart rate in normal healthy subject performing bicycle exercise: the sensor measured diastolic/systolic time ratio is >1 up to 140 beats per minute.

Lower panel: The curve of the diastolic force variation as a function of heart rate in a patient with heart failure performing bicycle exercise: a dramatic shortening of diastolic/systolic time ratio occurs with a reversal of the diastolic/systolic time ratio at 100 bpm heart rate. The diastolic force-frequency relation is defined normal when the peak stress diastolic time/systolic time ratio is positive (>1). The diastolic force-frequency relation is defined abnormal when the peak stress diastolic time/systolic time ratio is negative (<1). The diastolic force-frequency relation critical heart rate is defined as the heart rate beyond which the diastolic time/systolic time ratio from positive turns negative.

### Example 8

### Shown in FIG. 8

The Curve of the Second Cardiac Tone Vibration Amplitude as a Function of Heart Rate

X-axis: heart frequency bpm, (beats per minute); exercise=exercise in progress; recovery=recovery from exercise

Y-axis: second cardiac tone vibration amplitude, g×10⁻³ (g=9.8 m/sec²)

Computing the second heart sound amplitude variation as a function of heart rate. All the parameters are acquired as instantaneous values during exercise and recovery; continuous line=force mobile mean. Instantaneous force values scattering (points) depend on the respiratory cycle and thorax expansion.

### Example 9

### Shown in FIG. 9

X-axis: heart frequency bpm, (beats per minute). Y-axis: systemic blood pressure % (percent) changes assessed by second cardiac tone vibration amplitude % (percent) changes.

Given a baseline systemic pressure value, an algorithm, that uses the sensor outputs, allows the monitoring of the systemic pressure variations during stress/exercise/activity.

### Example 10

### Shown in FIG. 10

X-axis: time, seconds

Y-axis. Upper panel: LV volume, mL=left ventricular volume changes (peak=end-diastolic volume, nadir=end-systolic volume); LV pressure, mmHg=left ventricular pressure changes (peak=systolic pressure, nadir=diastolic pressure). Lower panel: cyclic first cardiac tone vibration amplitude, g (g=9.8 m/sec²).

According to the Frank-Starling law of the heart, the first cardiac tone vibration amplitude shows cyclic variations (a=lower amplitude; b=higher amplitude; c=lower amplitude; etc) mirroring the respiratory rate and depth of the respiratory system

### Example 11

### Shown in FIG. 11

X-axis: time, seconds.

Y-axis.

Upper panels: Aortic pressure, mmHg=cyclic aortic pressure changes (peak=systolic pressure, nadir=diastolic pressure).

Middle panels: dP/dt=left ventricular dP/dt changes (peak=positive max dP/dt, nadir=negative max. dP/dt) showing, left panel, normal isovolumic relaxation; right panel, blunted isovolumic relaxation.

Lower panels: sensor measured left ventricular active relaxation vibration amplitude, g×10⁻³ (g=9.8 m/sec²): left, a=normal left ventricular active relaxation vibration amplitude; right, b=blunted left ventricular active relaxation vibration amplitude.

### Example 12

### Shown in FIG. 12

X-axis: heart rate frequency bpm, (beats per minute); ex=exercise in progress; rec=recovery from exercise

Y-axis: first cardiac tone vibration amplitude, g×10⁻³ (g=9.8 m/sec²)

Continuous line=force mobile mean. Instantaneous force values scattering (points) depend on the respiratory cycle and thorax expansion;

The sensor measures the slope of the force-frequency relation during aerobic and anaerobic exercise. In the healthy heart, during the aerobic exercise the slope of the force-frequency relation is slightly up-sloping. During anaerobic exercise, in contrast to aerobic exercise, the slope of the force-frequency relation is much steeper, and the anaerobic threshold heart rate is quantified

### Example 13

### Shown in FIG. 13

The accelerometer sensor emitting the pulmonary artery pressure-frequency relationship continuously monitors the time gap between the aortic valve opening and pulmonary valve opening, and the time gap between aortic valve closure and pulmonary valve closure. In the normal heart the pulmonary valve opens before (a) and closes after (b) the aortic valve. When mild pulmonary hypertension occurs, the pulmonary valve opens (c) and closes (d) simultaneously with the aortic valve. When severe pulmonary hypertension occurs, the pulmonary valve opens before (e) and closes after (f) the aortic valve.

### Example 14

### Shown in FIG. 14

Upper panel: X-axis: heart rate frequency bpm, (beats per minute);

Y-axis: sensor derived systolic pulmonary artery pressure (mmhg)

Lower panel:

Given a basal rest value (preferably by inserting a baseline measured hemodynamic or Doppler derivative measure), an algorithm, based on the time gap between aortic valve and pulmonary valve opening and closure, easily calculates the pulmonary pressure values changes: a=normal pulmonary artery pressure at rest and during exercise; b=normal pulmonary artery pressure at rest and mild pulmonary hypertension during exercise; c=mild pulmonary hypertension at rest and severe pulmonary hypertension during exercise

### Example 15

### Shown in FIG. 15

X-axis: heart rate frequency bpm, (beats per minute);

Y-axis: force amplitude, g×10⁻³ (g=9.8 m/sec²)

Left panel The force-frequency relation in a patient with chronic heart failure (CHF) during exercise (full symbols) and during recovery from exercise (empty symbols) The post-exercise systolic force overshoot (defined as a relative increase in recovery systolic force of more than 10% with respect to the exercise/activity value) is recorded with the cutaneous sensor and is associated with an abnormal flat force-frequency relation during exercise: the contractile overshoot is a compensatory phenomenon in chronic heart failure.

Right panel: The force-frequency relation in a healthy subject (control) during exercise (full symbols) and during recovery from exercise (empty symbols) The force-frequency relation is normal up-sloping during exercise without a recovery overshoot.

### Example 16

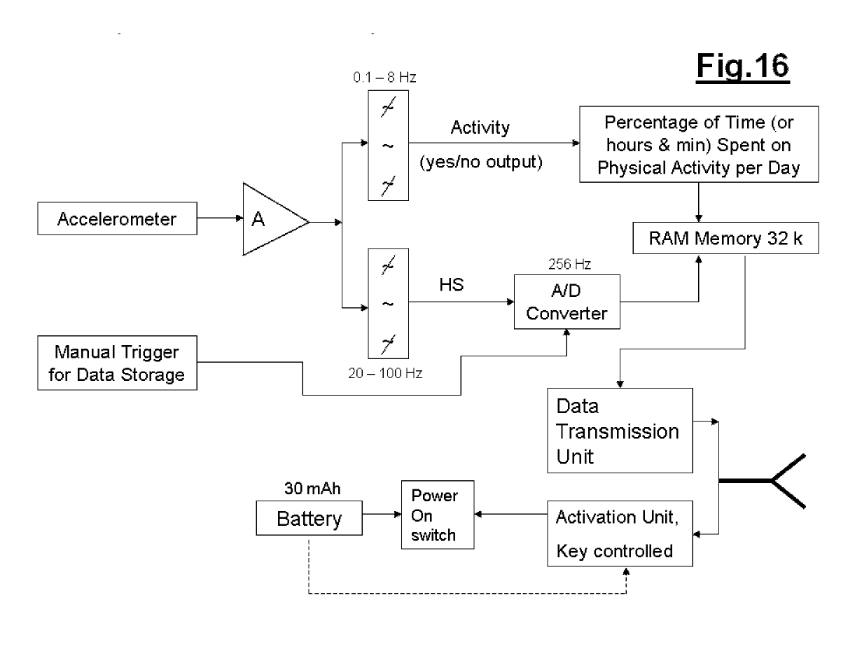

### Shown in FIG. 16

A block diagram illustrating an example configuration of a cardiac tone analyzer to monitor the individual force-frequency relation which further communicates with a remote service center

### Example 17

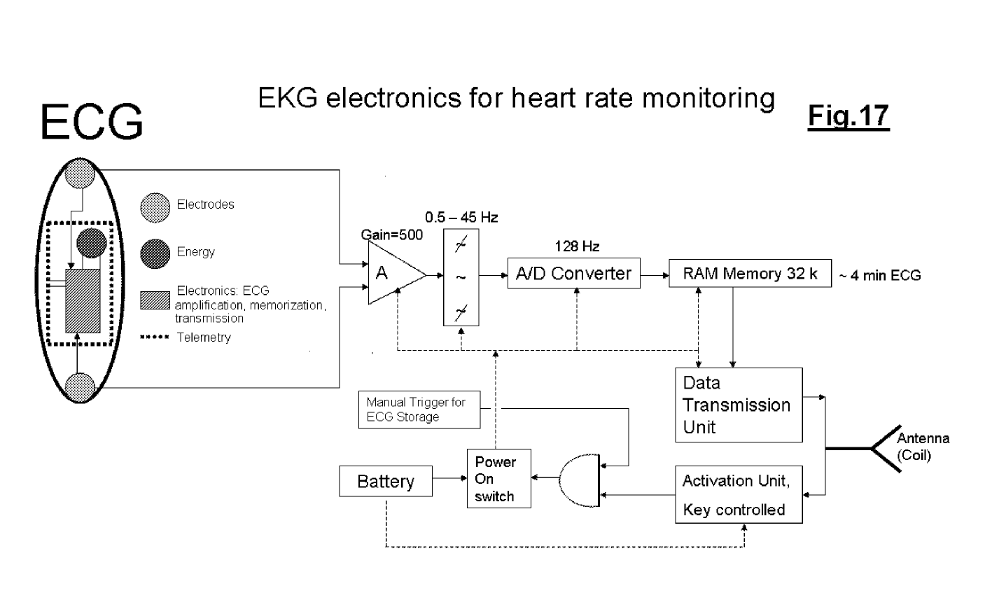

### Shown in FIG. 17

A block diagram of a circuit for detecting an ECG signal according to an embodiment of the present invention.

1. Method for quantifying and monitoring cardiovascular function comprising continuous determination of significant individual cardiovascular function parameters through at least one, operator-independent platform during a sample period at rest, the method comprising:
recording the data determined, continuously monitoring these data during pharmacological stress or exercise activity,
comparing the memorized data with those determined during the same time span of the sample period and comparing the cardiovascular function changes occurring during stress or exercise vs rest, and
comparing the cardiovascular function changes occurring during recovery vs rest and vs. stress or exercise, the memorized parameters comprising at least one curve of a cardiovascular function selected from the group consisting of: a systemic blood pressure-frequency relation, a force-frequency relation, a respiratory rate-frequency relation, a diastolic left ventricular active relaxation-frequency relation, a diastolic right ventricular active relaxation-frequency relation, a pulmonary artery pressure-frequency relation, an anaerobic threshold-frequency relation, a recovery contractility and a diastolic function overshoot-frequency relation, wherein the sensor is an accelerometer measuring the vibrations produced by the cardiovascular system and physiologically transferred from the inside of the body to the chest surface.

2. The method as claimed in claim 1, wherein the memorized parameters are the heart rate, activity level, cardiovascular function, and the curve of the cardiovascular function variation as a function of heart rate and/or activity.

3. The method as claimed in claim 2, further comprising expressing the cardiovascular function as a set of physiological parameters obtained by algebraic calculations through obtained measurements.

4. The method as claimed in claim 2, further comprising deriving effective measurements of the cardiovascular function-frequency relation by a multi-sensorial, simultaneous, monitoring strategy with a mobile priority intelligent algorithm; the mobile priority intelligent algorithm gives priority to the best sensor information in a time-mobile information flux and blind non-effective measurements to obtain continuously intelligible outputs of the cardiovascular function-frequency relation.

5. An apparatus for implementing the method as claimed in claim 1 comprising at least one accelerometer emitting cardiovascular function-indicative signals associated with an ECG sensor and/or an activity sensor arranged to measure cardiac electrical activity and/or body activity and to emit electrical signals indicative thereof wherein means are provided for transforming the signals emitted by said sensors from analog to digital, and to feed the signals to a processor to obtain a cardiovascular function-frequency relation curve, and the variations thereof over time, a memory for memorizing the digital data in an ordered manner being associated to the processor, the memorized data can be also read remotely by a telemetric connection.

6. The apparatus as claimed in claim 5, wherein the accelerometer monitors a systolic force-frequency relation.

7. The apparatus as claimed in claim 5, wherein the accelerometer monitors a diastolic force-frequency relation and filling function of the heart.

8. The apparatus as claimed in claim 5, wherein the accelerometer monitors a systemic blood pressure-frequency relation.

9. The apparatus as claimed in claim 5, wherein the accelerometer is configured to assess a respiratory rate and function, and monitors respiratory-frequency relation.

10. The apparatus as claimed in claim 5, wherein the accelerometer provides information on active relaxation of a left ventricle in diastole trough monitoring a pressure gradient across an aortic valve at the time of closure and the left ventricle and giving information on active relaxation of the right ventricle in diastole trough monitoring the pressure gradient across the pulmonary valve at the time of closure and the right ventricle, and for the monitoring of the diastolic left and right ventricular active relaxation-frequency relation.

11. The apparatus as claimed in claim 5, wherein the accelerometer monitors the anaerobic threshold-frequency relation.

12. The apparatus as claimed in claim 5, wherein the accelerometer monitors continuously a time gap between the aortic valve opening and pulmonary valve opening, and a time gap between aortic valve closure and pulmonary valve closure; providing information on pulmonary artery pressure, and the pulmonary artery pressure-frequency relationship.

13. The apparatus as claimed in claim 5 wherein sensor emitting cardiovascular function-indicative signals is a vibration sensor configured to detect a contractility overshoot and/or a diastolic function overshoot in a post stress or post-exercise recovery period.

14. The apparatus as claimed in claim 5, wherein the accelerometer is associated with an ECG sensor and is configured to measure cardiac electrical activity and to emit electrical signals indicative thereof and is associated with an activity and position sensor to evaluate activity or body position.

15. The apparatus as claimed in claim 5, further comprising at least one microprocessor, with at least one sensor, and at least one calculation program to derive the cardiovascular function-frequency relation, configured to automatically calculate a set of physiological parameters, using data obtained by the sensor, or data quantified by standard methods and digitized by operators, or both sensor data and standard quantified data, creating a cardiovascular function-frequency relationship; at least one of the automatically calculated parameters are selected from the group consisting of: body surface area, stroke volume, cardiac output, mean arterial pressure, pulse pressure, end systolic pressure, LV elastance (Eₑₛ) index, effective arterial elastance index (Eai), ventricular-arterial coupling, systemic vascular resistance, systemic arterial compliance, mitral E/E′, diastolic mean filling rate, diastolic time/systolic time ratio, pulmonary artery systolic pressure, pulmonary artery end-diastolic pressure, mean pulmonary pressure, pulmonary artery pulse pressure, pulmonary vascular resistance, pulmonary vascular capacitance, and pulmonary capillary wedge pressure (PCWP); at rest, during stress or physical activity, in recovery period, as values or value changes vs rest.
